(19) United States
(12) Patent Application Publication
(10) Pub. No.: US 2007/0239225 A1
(43) Pub. Date: Oct. 11, 2007

(54) Training device and method to suppress sounds caused by sleep and breathing disorders

(76) Inventor: John H. Saringer, Stouffville (CA)

Correspondence Address: Ralph A. Dowell of DOWELL & DOWELL P.C., 2111 Eisenhower Ave, Suite 406, Alexandria, VA, 22314, US

(21) Appl. No.: 11/710,619

(22) Filed: Feb. 26, 2007

Related U.S. Application Data
(60) Provisional application No. 60/776,961, filed on Feb. 28, 2006.

(51) Int. Cl. A61N 1/36; A61B 5/22

(52) U.S. Cl. 607/42; 600/529

(57) ABSTRACT

A training method and device to suppress sounds caused by sleep and breathing disorders, such as, snoring and sleep apnea. The system records the actual snoring of the subject, modifies it, by time delay and perhaps other sound modifying techniques, and broadcasts it to the subject by a speaker or earphone. This trains the person to attend to his/her own snoring, so that he/she might stop it by moving parts of his/her body or changing breathing patterns. The method and device may include the combination and/or integration of hearing aid functionality and means for detection of snoring sounds.

### CROSS REFERENCE TO RELATED APPLICATION

This patent application is related to, and claims priority from, U.S. provisional patent application Ser. No. 60,776,961 filed on Feb. 28, 2006, entitled TRAINING DEVICE AND METHOD TO SUPPRESS SOUNDS CAUSED BY SLEEP AND BREATHING DISORDERS, SUCH AS SNORING AND SLEEP APNEA, filed in English, which is incorporated herein by reference in its entirety.

### FIELD OF THE INVENTION

The present invention relates to methods and biofeedback training devices for the suppression of snoring sounds and sounds associated with sleep or breathing disorders, such as sleep apnea.

### BACKGROUND AND SUMMARY OF THE INVENTION

Almost all people who snore are unable to hear their own snoring. The brain has a way of blocking the sound, making it tolerable for the subject, even while it becomes intolerable for another person trying to sleep in the same room. Once a subject learns to ignore their own snoring sounds, the sounds may then further increase without disturbing the subject, however such increased habituation can make the snoring even more intolerable for anyone else attempting to sleep in the same room. In contrast, the brain is vigilant in detecting unfamiliar sounds even while the subject is sound asleep. It is the purpose of this invention to reverse this adaptation by making the subject self aware of their snoring and compelling the subject to learn a quieter breathing pattern by feeding back the snoring sound in such a manner that the subject attends to it. Various devices for the suppression of snoring have been proposed which disturb or punish the subject when they snore loudly by an auditory alarm, vibration, electric shock or other noxious stimuli. Some of them, such as that disclosed in U.S. Pat. No. 5,477,867 emit modulated sounds to train the subject by increasing the noxious feedback in proportion to increasing loudness of the snoring. The difficulty with all these devices is that they do not give accurate feedback and the subject easily habituates to the stimulus, rendering these methods of limited effectiveness. The difficulty with some of these devices that include an earpiece is that they may prevent the subject from hearing ambient sounds that might alert them to danger.

Therefore, what is needed is a method and device which teaches the subject to attend to their own snoring and unconsciously stop it by, for example, moving their head or moving their mouth or throat, or changing breathing patterns all while still asleep at some level. Further, such a method or device should not be subject to accommodation or habituation and at the same time should allow the subject to hear ambient sounds, even if the subject has hearing impairment.

### SUMMARY OF THE INVENTION

This present invention provides methods and devices which trains a subject who snores to become self aware that they are snoring by bypassing the brain's habituation mechanism which would otherwise block the snoring sound. To train awareness of snoring, in an embodiment of the invention the subject's own snoring sounds are used as the prompt, and modified only to that degree that allows them to side-step the mind's normal ability to ignore them. The snoring sounds that are used for the prompt may be those that were vocalized by the subject some time interval before, usually a fraction of a second before which are played back to the subject after a suitable time delay so that the subject's brain thinks these sounds were not produced by the subject. Alternatively, after detection of sleep induced sounds pre-recorded sounds not from the user may be played back without a time delay and the brain will interpret these as not being produced by the subject.

Thus in one aspect of the invention there is provided a device for training a person to suppress snoring sounds and/or sounds associated with sleep or breathing disorders, comprising:

a) a microprocessor controller, a detector to detect sound signals emitted by a person during periods of sleep in communication said microprocessor controller so that sound signals emitted by the person and detected by said detector are converted to sound signals recorded by said microprocessor controller;

b) a speaker in communication with said microprocessor; and

c) said microprocessor controller including processing means configured to
i) prepare pre-selected sound signals that the person's brain interprets as having originated outside of the person and not produced by the person to prepare, and
ii) transmit said pre-selected sound signals to said speaker after said detector detects the sounds emitted by the person.

The present invention also provides a device for training a person to suppress snoring sounds and/or sounds associated with sleep or breathing disorders, comprising:

a) a microprocessor controller, a detector to detect sound signals emitted by a person during periods of sleep in communication said microprocessor controller so that sound signals emitted by the person and detected by said detector are converted to sound signals recorded by said microprocessor controller;

b) a speaker in communication with said microprocessor; and

c) said microprocessor controller including processing means configured to
ii) transmit said recorded sound signals to said speaker after a pre-selected time delay from when the sound signals emitted by the person are first detected.

In another aspect of the present invention there is provided a method for training a person to suppress snoring sounds and/or sounds associated with sleep or breathing, comprising:

a) detecting sound signals emitted by a person during periods of sleep; and thereafter

b) playing pre-selected sound signals within earshot of the person with the sound pre-selected sound signals being selected such that the person's brain interprets the pre-selected sound signals as having originated outside of the person and not produced by the person.

A further understanding of the functional and advantageous aspects of the invention can be realized by reference to the following detailed description and drawings.

### BRIEF DESCRIPTION OF THE DRAWINGS

Preferred embodiments of the invention will now be described, by way of example only, with reference to the drawings, which are not to scale, in which:

FIG. 1 is a perspective frontal view of a training device for the suppression of snoring sounds and sounds associated with sleep or breathing disorders constructed in accordance with the present invention in the form of an earplug;

FIG. 2 is a sectional view of the earplug device of FIG. 1 in the outer ear of a subject;

FIG. 3 shows an embodiment of the earplug device showing the internal components;

FIG. 4 is a of another embodiment of the earplug training device according to the present invention;

FIG. 5 is a perspective view of a remote control unit, which may operate in cooperation with separate elements of the system, or act as a stand-alone device; and

FIG. 6 is a perspective view of another embodiment of the training device in the form of a pillow.

### DETAILED DESCRIPTION OF THE INVENTION

Generally speaking, the systems described herein are directed to methods and devices for the suppression of snoring sounds and sounds associated with sleep or breathing disorders, such as sleep apnea. As required, embodiments of the present invention are disclosed herein. However, the disclosed embodiments are merely exemplary, and it should be understood that the invention may be embodied in many various and alternative forms. The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to a method and devices for the suppression of snoring sounds and the like.

As used herein, the term “about”, when used in conjunction with ranges of dimensions, temperatures or other physical properties or characteristics is meant to cover slight variations that may exist in the upper and lower limits of the ranges of dimensions so as to not exclude embodiments where on average most of the dimensions are satisfied but where statistically dimensions may exist outside this region.

The present invention provides a method and devices for training a subject to stop or reduce their own snoring. Without being bound by any theory, this is accomplished by the method and device disclosed herein by recording when snoring events (or other sound events) start and then playing back sounds which would not be recognized by the subject as being sounds they have made. This may be achieved in several ways including playing back to the subject the same sounds emitted by the subject but with a time delay long enough so that the subject will generally not recognize them as their own. Other ways of achieving this may include simply playing back pre-recorded sounds or synthesized sounds which would not be recognized as sounds emitted by the subject and this playback could be done without the same time delay since clearly these sounds would not be recognized by the subject's brain as being their own. Another way is to record the subject's own sounds and manipulating them and playing them back without the above mentioned time delay (or they could be played back with the time delay).

Another way to achieve this is to produce a small time delay between the subjects ears picking up the sounds. This may be done using two ear pieces which are programmed to emit the sounds at different times into the subject's ears. Alternatively, placing a sound source a sufficient distance from the subject and emitting sounds will be interpreted by the subject's brain as being sounds not emitted by the subject since the sounds will be detected at slightly different times by each ear because of the difference in distance and intensity of the sounds picked by the subjects two different ears.

Thus, in the cases where the method involves both ears detecting the sounds it is advantageous if one ear receives a slightly shifted signal from the other so that the sound is perceived to come from outside the body. Prolonged use of the device alters the breathing and sleeping habits of the subject so as to reduce snoring. Occasional use of the device after the initial retraining period may be necessary to maintain the new quieter sleep patterns and prevent relapse.

For some embodiments the device could be configured so that the system recognize when snoring or sleep disorder events are occurring, and this is accomplished by various methods, such as a simple volume threshold or more complex pattern recognition and/or including volume, frequency and time intervals; perhaps enhanced by: motion and/or level detection means, and/or means to detect darkness (in the bedroom). These embodiments use this information to modify the functionality of the system. For example, in those embodiments that include hearing aid functionality, the detection that the subject is not snoring, might suppress the anti-snoring function and optimize the system to perform as a hearing aid; and when snoring is detected, the system might optimize for snoring detection and abatement, perhaps degrading or turning off the hearing aid function.

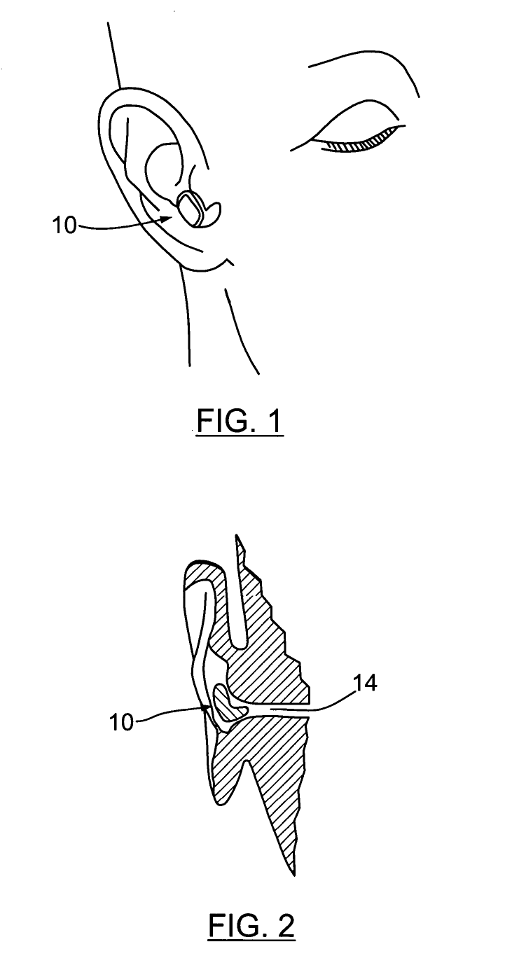

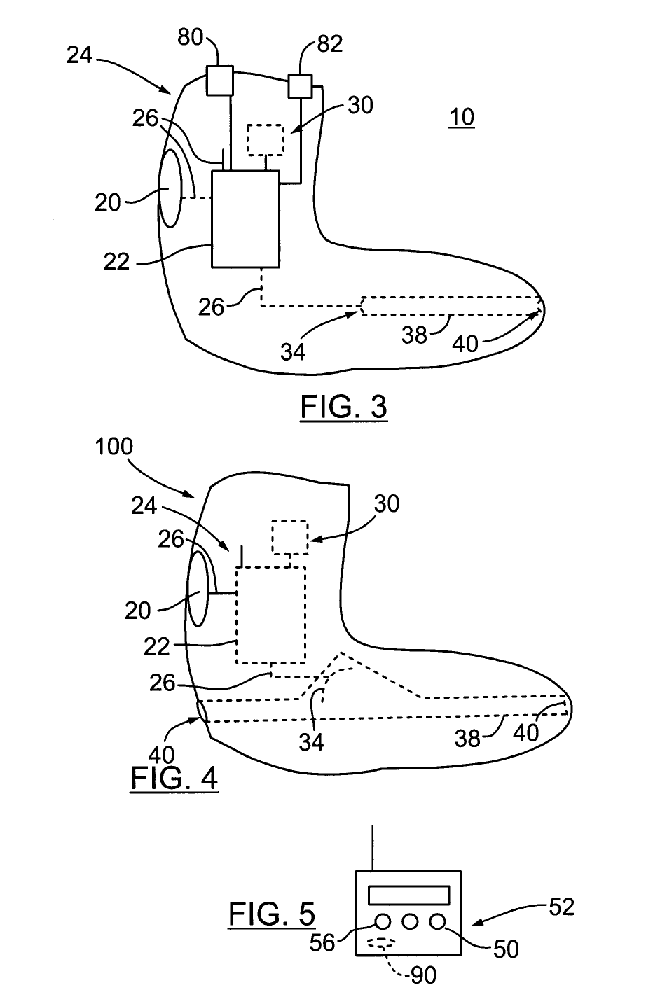

FIG. 1 and FIG. 2 illustrate an embodiment of the present invention in the form of a hearing aid earpiece (or earpiece) 10 which has a shape allowing it to be conveniently placed in the outer ear of one or both ears 14 of a subject 16. As illustrated in FIG. 3, the earpiece unit 10 houses a sound detector such as a microphone 20 for connected to a control unit 22, either wirelessly by an aerial 24 or by connector 26. A battery 30, rechargeable battery or other energy providing device powers the control unit 22. The microphone 20 detects the snoring sound and reports it to the controller, usually comprised of a computer and memory device, which manipulates the received signal by altering the output to a speaker 34, by delaying its broadcast, and/or modulating its frequency, amplitude, or adding or subtracting sounds. The modified sound is then transmitted to the speaker 34, wirelessly or by electrical connections 26, where it is broadcast through the channel 38, through orifice 40 into the ear canal 14 of one or both ears (FIG. 2). When two earpieces are used concurrently the signals to each ear may be varied in such a way so as to create the illusion that the signal originates from outside the body.

FIG. 4 is a perspective view of another embodiment of an earpiece 100 which is very similar to the earplug 10 in FIG. 3 except that the channel 38 extends through the earplug with two open ends 40. This allows ambient sounds to be detected by the subject. This feature allows the subject to hear ambient sounds, even when the unit 100 is off. This feature may make the unit safer for the wearer, since the sound of an intruder, or other dangerous occurrence, might otherwise go unnoticed.

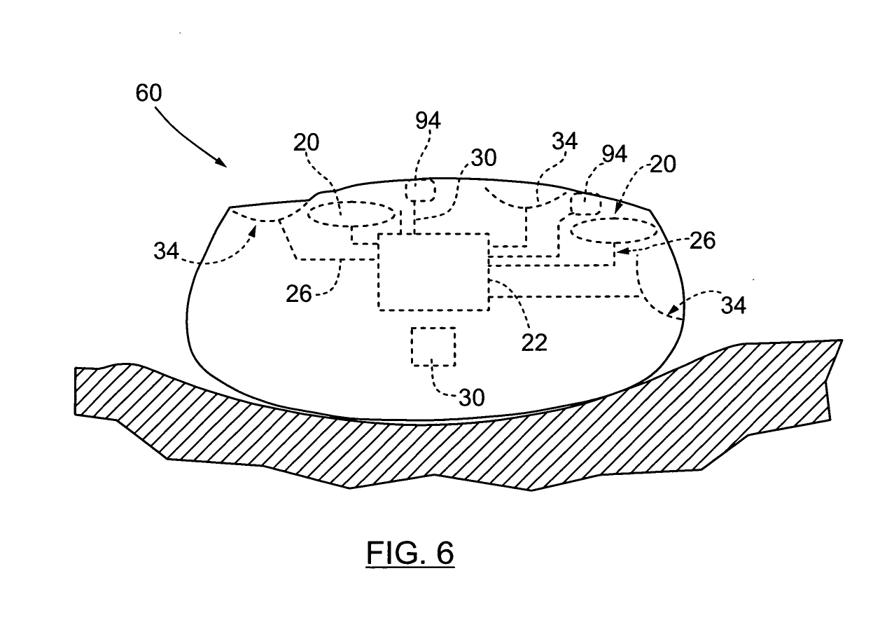

FIG. 5 shows a remote control which may be used to control one or two earpieces wirelessly or through a cord connection, and may incorporate some of the functional features of the earpieces so as to reduce the complexity of the earpieces. It may be preferred not to include or integrate controls 50 and 56 into the earpiece 10 directly because the size of these controls may be large compared to the earpiece. Similarly, functional processing steps may be more easily carried out by the control unit 52 than by the earpiece since the control unit is not constrained by size and power availability as are the earpieces. It will be recognized that the redistribution of functional elements between the control unit 52 and ear pieces 10 can improve the functionality of the whole system without changing the basic approach.

Indeed, the control unit 52 can be made to incorporate most if not all of the elements of the system if it is advantageous to do so. For example, when the control unit has a built in microphone 90 and speaker 34, it may operate independently and without need of earpieces, or alternatively broadcast through a remote speaker located in a pillow or other convenient location, or an earpiece containing just a speaker may be used. When sound signals are transmitted from control unit 52 the unit would include wireless transmitter.

Thus one device may combine the functionality of an anti-snoring device and a hearing aid by use of an electromechanical switch 50 on the remote control unit 52 or on the earpiece 10 or both, which can select the appropriate mode of operation, on and off, and between hearing aid simple, or anti-snoring device, or anti-snoring device with hearing aid acting simultaneously. In these embodiments, for example, the remote control unit 52 can also control the various other aspects of these modes, such as volume, tonal qualities, pitch and filtering options, thresholds for activation of anti-snoring functions etc, by selector 56.

In these embodiments, the controller 22 and/or 52 generally control both functions, and the microphone 20 and speaker 34 serve as such for both the anti-snoring and hearing aid. That is, the hearing aid uses the microphone 20 to pick up the ambient sounds, the controller 22 and/or 52 process that sound, such as amplifying it and filtering it, and the sound so processed is transmitted to the speaker 34, which broadcasts the sound, to the inner ear canal 14. The anti-snoring functions, whether alone, or in combination with hearing aid functionality, utilize the said components, microphone 20, controller 22 and/or 52, and speaker 34, as described above. It is to be understood however that in some preferred embodiments, the system may contain any number of microphones 20 and speakers 34, each with particular sonic qualities.

The controllers 22 and 52 may be configured to filter the input snoring sounds, and using pattern recognition algorithms, identify particular sounds that are symptomatic of particular conditions, and selectively feed back only those sounds, or preferentially amplifies only those sounds, after being modified as described above. For example, a subject suffering from sleep apnea may have particular sounds that result from breathing patterns associated with that condition, in addition to regular snoring so that the device may isolate just those sleep apnea related sounds, and feed these back to the subject, with the modifications described above, to specifically treat the sleep apnea condition.

It has be found that a suitable time delay for most subjects is between approximately one tenth of a second to approximately two seconds, with best results achieved with a delay of approximately one-quarter to half of a second. However, for subjects having varying responses, the time delays may be less than and/or greater than those delays in the range just referred to, and all these are considered to fall within the ambit of this invention.

The device may be configured to allow for a variable time delay in order to prevent accommodation or habituation, randomly or according to a schedule. Some preferred embodiments also, or alternatively, modulate the frequency and/or amplitude of the normal snoring sounds to assist the subject in attending to the rebroadcast sounds. Other preferred embodiments may add or subtracts sounds, or make other modifications to the original snoring sound sufficient for the purpose. Again, all of these modifications may be altered randomly or according to a schedule to prevent accommodation or habituation. Similarly, these modifications may be altered randomly or according to a schedule based on the effectiveness of the subject's response, using an adaptive algorithm.

When two earpieces are used, one in each ear, still greater effectiveness can be achieved by having each earpiece deliver a slightly different time delay. Small timing differences between each ear are usually interpreted by the brain as a sound originating from outside the body in a particular direction and hence are more likely to bypass the brain's mechanism to ignore the sound, particularly while asleep. The greatest effectiveness is achieved when the feedback is tuned to be indistinguishable from a sound originating from outside the body, and most unlike a sound originating from the body. This exploits the brain's innate alertness, while asleep, to external noises while bypassing its automatic suppression of self generated noise.

The microphone 20 may be configured to detect the subject's snoring and rather than time shifting or altering the sound of the subject's own snoring, simply plays back a prerecorded or synthesized snoring sound, for the duration of the subject's snoring, perhaps altering this rerecorded or synthesized snoring in a random, preprogrammed manner, and/or adaptive manner. This prerecorded sound may be the subject's own snoring sounds, the snoring sounds of another subject and/or synthesized snoring sounds. The differences between the subject's own snoring and the prerecorded snoring or the synthesized snoring will be sufficient that the subject will then attend to his own snoring.

The device may be configured to approximately match the volume or other parameters of the subject's snoring, perhaps with a time delay, with the preprogrammed or synthesized snoring. While preferred embodiments of the type referred to in this paragraph may not be quite as effective at those that alter the subject's own snoring, in real-time, as referred to in other paragraphs of this disclosure, it may be less expensive to implement, as the control units 22/52 need only have in memory a recording of a snoring routine, which may be looped, or the precursors or program instructions, from which the synthesized sounds are produced. In these embodiments, as in some of the other embodiments of the invention, the subject's own snoring may also be broadcast on the speaker 34, and/or be heard by the subject through a channel 38 (FIG. 4).

It should be noted that the preferred embodiments of this invention treat, not just snoring, but all sleep abnormalities that cause loud sounds such as sleep apnea.

Since the device includes a microphone 20, speaker 34 controller 22, which includes signal amplifiers, all incorporated into an earplug 10, (perhaps with remote control unit 52) it can be readily appreciated that these embodiments can also act as hearing aids. Therefore it is to be understood that some embodiments of the earplug 10 may act only as a snoring device, or as both a snoring device and hearing aid, and still fall within the scope of the present invention. For example standard hearing aids can be modified to remove the standard operating program in the control chip and a program inserted which will implement the present method such as emitting pre-recorded sounds, manipulate the subject's snoring sounds such that they are unrecognizable by the subject, by playing back the subjects own sounds with the time delay. For embodiments using two modified hearing aids they can be produced in pairs with each device programmed to play the sounds in a differential manner as discussed above such that the subject's brain interprets the sounds as being from an outside source.

Alternatively, existing hearing aids may have, in addition to the regular control programming, additional control programming added to implement the present method. Small mechanical switches could be used to switch between the hearing aid mode and the biofeedback mode. Switching could also be achieved using pattern recognition programs and/or external sources (position, light etc.) which determine when the subject is asleep.

The fact that both functions can be incorporated into one device greatly reduces the cost of the snoring feature for those that also suffer from hearing disabilities and require a hearing aid. For those that wish to leave the hearing aids in their ears during sleep, they will now be able to use the anti-snoring device at the same time. The integration of these functions is also safer for those subjects that have hearing disabilities and wish to be alerted to unusual sounds, which might signal impending danger, during the night.

Where the functionality of the hearing aid and anti-snoring are combined and/or integrated, it may be important that the system recognize when the person is sleeping and/or snoring, and for this purpose pattern recognition software/hardware that recognizes snoring be included into the controller 22 and/or remote control unit 52.

In embodiments which do not use a remote controller, any control settings are made on the hearing aid ear piece, using standard electromechanical controls 80, 82. In other embodiments of the invention the remote control unit 52 may contain, or be connected to, a speaker that transmits the modified sound near the subject, rather than to an earplug. It is to be understood that the principal components of the system can be arranged and located in any convenient way. For example some or all of the principal components may be incorporated into a pillow 60 as illustrated on FIG. 6. In addition to a pillow, the components could be incorporated into a hat or cap, or a head band.

In another embodiment of the invention, the microphone 20 and/or the speaker 34 may be located remote to the earplug 10 or pillow 60. For example, as illustrated in FIG. 5, in the case of the earplug 10, the microphone 90 might be placed in the remote control unit 52 which permits the system to operate, even if the subject has his head on the pillow such that the earplug 10 is covered by the pillow 60, which would otherwise prevent a microphone 20 in the earplug 10 from detecting the sound. Other embodiments have a remote control unit 52 that includes a speaker 34 as well as the microphone 90 and can act as a stand-alone unit, without an earplug, as illustrated in FIG. 5, but this arrangement might also awake a person sharing the same room.

An example of a simple preferred embodiment of the invention would be a portable computer (PC) or a personal digital assistant (PDA) 52/22 that incorporates PC functionality which runs a time delay software program, such as “AudioSyncer” sold by Matthews Software, 26150 Greythorne Trail, Farmington Hills, Mich., 48334, U.S.A. (matthewssoftware.com). Such a system including a microphone 20 and earplug or speaker in a pillow 60, would compose a simple preferred embodiment of the invention.

FIG. 6 illustrates the system incorporated into a pillow, but it is to be understood that it may be incorporated in whole or in part in any convenient article or object, such as a headband or headboard and still be within the ambit of the invention. As noted above, some of the principal parts of the system may be remote, such as the remote control unit 52. In the preferred embodiment illustrated in FIG. 6, the speaker 34 is located inside the pillow near where the ear of the subject would be normally located. In some preferred embodiments, any number of speakers 34 and microphones 20 may be utilized and in some preferred embodiments, sensors may be utilized to locate the mouth and ear of the subject and activate only those microphones and speakers closest to the subject's mouth and ear, respectively. For example, some preferred embodiments have the controller 22 compare the volume inputs from the multiple microphones 20 to locate the mouth and/or in combination with such sensors as pressure, heat, motion or sound sensors (transducers) 44 that locate the ear and mouth, so that the controller can send the signal to that speaker 34, that is closest to the ear of the subject.

The control unit 22 and/or the remote control unit 52 can simply detect, record, modify (with time delay etc.) and transmit all the sounds that are detected by the microphone 20, or only the snoring sounds. The transmission to the speakers of all sounds detected may be sufficient, where the microphone is designed to detect substantially that sound only, or may be convenient where simplicity is required. Other preferred embodiments include electronic means of filtering the sound signal such that only the modified snoring sounds are passed to the speaker 34.

Tests by the inventor show that retraining with the present method and devices can take several months, with substantial gains typically occurring within three (3) weeks. Sleep is usually greatly disturbed in the first few nights of use until breathing sounds begin to come under control and then diminish thereafter as quieter sleep patterns are established.

While the present invention has been described in conjunction with preferred embodiments, it is to be understood that modifications and variations may be resorted to without departing from the spirit and scope of the invention as those skilled in the art will readily understand. Such modifications and variations are considered to be within the purview and scope of the inventions and appended claims.

As used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

Therefore what is claimed is:

1. Device for training a person to suppress snoring sounds and/or sounds associated with sleep or breathing disorders, comprising:
a) a microprocessor controller, a detector to detect sound signals emitted by a person during periods of sleep in communication said microprocessor controller so that sound signals emitted by the person and detected by said detector are converted to sound signals recorded by said microprocessor controller;
b) a speaker in communication with said microprocessor; and
c) said microprocessor controller including processing means configured to
i) prepare pre-selected sound signals that the person's brain interprets as having originated outside of the person and not produced by the person to prepare, and
ii) transmit said pre-selected sound signals to said speaker after said detector detects the sounds emitted by the person.

2. The device according to claim 1 wherein said microprocessor controller, detector and speaker are located in a hearing aid ear piece having a size and shape such that the training device can be inserted into a user's ear, and wherein said detector and speaker are electrically connected to said microprocessor controller.

3. The device according to claim 1 wherein said microprocessor controller, detector and speaker are located in a pillow, and wherein said detector and speaker are electrically connected to said microprocessor controller.

4. The device according to claim 1 wherein said microprocessor controller, detector and speaker are located in a cap, headband or headgear, and wherein said detector and speaker are electrically connected to said microprocessor controller.

5. The device according to claim 1 wherein said pre-selected sound signals are the recorded sound signals, and wherein said processing means is configured to play the recorded sound signals after a pre-selected time delay.

6. The device according to claim 1 wherein said processing means is configured to manipulate the recorded sound signals to produce said pre-selected sound signals.

7. The device according to claim 1 wherein said processing means is configured to manipulate the recorded sound signals to alter any one or combination of modulating frequency of the recorded sound signals, modulating amplitude of the recorded sound signals, adding additional sound signals to the recorded sounds and subtracting selected sound signals from the recorded sound signals to produce said pre-selected sound signals.

8. The device according to claim 6 wherein said processing means is configured to manipulate the recorded sound signals randomly or according to a schedule.

9. The device according to claim 8 wherein said processing means is configured to record the person's response to the playing of the pre-selected sound signals, and wherein said processing means includes an adaptive algorithm for producing said schedule, and wherein said schedule is prepared using said adaptive algorithm and is based on the effectiveness of the subject's response.

10. The device according to claim 5 wherein said processing means is configured to provide a variable time delay, randomly or according to a schedule.

11. The device according to claim 10 wherein said processing means is configured to record the person's response to the playing of the pre-selected sound signals, and wherein said processing means includes an adaptive algorithm for producing said schedule, and wherein said schedule is prepared using said adaptive algorithm and is based on the effectiveness of the subject's response.

12. The device according to claim 1 wherein said microprocessor controller includes volume control means for adjusting a volume of the replayed sound signals.

13. The device according to claim 1 wherein said processing means includes sound recognition programs to recognize when snoring or other sleep induced sound events are occurring.

14. The device according to claim 1 including any one or combination of motion detection means, and level detection means in communication with said microprocessor controller to detect movement of the person, and light detection means in communication with said microprocessor controller for detecting ambient light levels.

15. The device according to claim 1 in wherein said speaker is a first speaker directed toward one ear and including a second speaker connected to said microprocessor controller, and wherein said processing means is configured to play said pre-selected sound signals to the first and second speakers but with a time delay between them.

16. The device according to claim 15 wherein said processing means is configured to play said pre-selected sound signals from the first and second speakers but with a volume difference.

17. The device according to claim 1 retrofitted into a hearing aid, including switching means for switching between a hearing aid function during waking hours and a training device during sleeping hours.

18. The device according to claim 1 wherein said microprocessor controller, detector and speaker are located in a housing spaced from the person but within earshot of the person, and wherein said detector and speaker are electrically connected to said microprocessor controller.

19. The device according to claim 1 wherein said microprocessor controller and detector and are located in a housing spaced from the subject, and wherein the speaker is located in a hearing aid earpiece, including a wireless transmitter located in said housing for transmitting the pre-selected sound signals to the hearing aid earpiece.

20. A method for training a person to suppress snoring sounds and/or sounds associated with sleep or breathing, comprising:
a) detecting sound signals emitted by a person during periods of sleep; and thereafter
b) playing pre-selected sound signals within earshot of the person with the sound pre-selected sound signals being selected such that the person's brain interprets the pre-selected sound signals as having originated outside of the person and not produced by the person.

21. The method according to claim 20 wherein said step of detecting sound signals emitted by a person during periods of sleep includes recording said sound signals emitted by the person, and wherein said pre-selected sound signals played are the recorded sound signals emitted by the person which are played after a pre-selected time delay after the sound signals emitted by the person are detected.

22. The method according to claim 21 wherein said pre-selected time delay is in a range from about one tenth of a second to about two seconds.

23. The method according to claim 20 wherein said step of detecting sound signals emitted by a person during periods of sleep includes recording said sound signals emitted by the person, including manipulating the recorded sound signals in order to distort the recorded sound signals such that they do not sound like the original sound signals, and wherein said pre-selected sound signals played are the manipulated recorded sound signals emitted by the person.

24. The method according to claim 23 wherein said step of manipulating the recorded sound signals includes any one or combination of modulating frequency of the recorded sound signals, modulating amplitude of the recorded sound signals, adding additional sounds to the recorded sound signals and subtracting selected sound signals to the recorded sounds.

25. The method according to claim 24 wherein said step of manipulating the recorded sound signals is done randomly or according to a schedule.

26. The method according to claim 25 including observing the person's response to the playing of the pre-selected sound signals, wherein said schedule is based on the effectiveness of the subject's response, using an adaptive algorithm.

27. The method according to claim 23 wherein said step of manipulating the recorded sound signals includes electronically filtering the recorded sound signals to achieve the desired sound signals that are played.

28. The method according to claim 21 including observing the person's response to the playing of the pre-selected sound signals, and varying said selected period of time delay, randomly or according to a schedule.

29. The method according to claim 28 wherein said schedule is based on the effectiveness of the person's response to the playing of the pre-selected sound signals using an adaptive algorithm.

30. The method according to claim 21 wherein the pre-selected sound signals are pre-recorded snoring sounds.

31. The method according to claim 30 wherein said pre-recorded snoring sounds are any one or combination of the person's own snoring sounds, the snoring sounds of another person and synthesized snoring sounds.

32. The method according to claim 30 including a step of altering the pre-recorded snoring sounds in any one of a random, preprogrammed, and adaptive manner.

33. The method according to claim 20 wherein said steps of detecting sound signals and playing pre-selected sound signals are performed using a hearing aid ear piece containing a microphone, speaker and microprocessor connected to said microphone and speaker, said microprocessor including processing means programmed to transmit said pre-selected sound signals to said speaker a pre-selected time delay after being received by said microphone, said hearing aid ear piece being inserted into one of the person's ears.

34. The method according to claim 33 wherein said steps of detecting sound signals and playing pre-selected sound signals are performed using a first and second hearing aid ear piece each containing a microphone, speaker and microprocessor connected to said microphone and speaker, said microprocessor including processing means programmed to transmit said pre-selected sound signals to said speaker a pre-selected time delay after being received by said microphone, said first and second hearing aid ear pieces being each inserted into one of the person's ears.

35. The method according to claim 34 wherein said processing means in the first and second hearing aid ear pieces are programmed to play said pre-selected sound signals after different time delays.

36. Device for training a person to suppress snoring sounds and/or sounds associated with sleep or breathing disorders, comprising:
a) a microprocessor controller, a detector to detect sound signals emitted by a person during periods of sleep in communication said microprocessor controller so that sound signals emitted by the person and detected by said detector are converted to sound signals recorded by said microprocessor controller;
b) a speaker in communication with said microprocessor; and
c) said microprocessor controller including processing means configured to
ii) transmit said recorded sound signals to said speaker after a pre-selected time delay from when the sound signals emitted by the person are first detected.

37. The device according to claim 36 wherein said pre-selected time delay is in a range from about one tenth of a second to about two seconds.

38. The device according to claim 36 wherein said microprocessor controller, detector and speaker are located in a hearing aid ear piece having a size and shape such that the training device can be inserted into a user's ear.

39. The device according to claim 36 wherein said processing means is configured to manipulate the recorded sound signals to produce said pre-selected sound signals.

40. The device according to claim 36 wherein said processing means is configured to manipulate the recorded sound signals to alter any one or combination of modulating frequency of the recorded sound signals, modulating amplitude of the recorded sound signals, adding additional sound signals to the recorded sounds and subtracting selected sound signals from the recorded sound signals to produce said pre-selected sound signals.

41. The device according to claim 39 wherein said processing means is configured to manipulate the recorded sound signals randomly or according to a schedule.

42. The device according to claim 41 wherein said processing means is configured to record the person's response to the playing of the pre-selected sound signals, and wherein said processing means includes an adaptive algorithm for producing said schedule, and wherein said schedule is prepared using said adaptive algorithm and is based on the effectiveness of the subject's response.

43. The device according to claim 36 wherein said processing means is configured to provide a variable time delay, randomly or according to a schedule.

44. The device according to claim 43 wherein said processing means is configured to record the person's response to the playing of the pre-selected sound signals, and wherein said processing means includes an adaptive algorithm for producing said schedule, and wherein said schedule is prepared using said adaptive algorithm and is based on the effectiveness of the subject's response.

45. The device according to claim 36 wherein said processing means includes sound recognition programs to recognize when snoring or other sleep induced sound events are occurring.
